(19) United States
(12) Patent Application Publication
(10) Pub. No.: US 2024/0304309 A1
(43) Pub. Date: Sep. 12, 2024

(54) Method And System For Assessing Nonalcoholic Steatohepatitis

(71) Applicant: Histoindex Pte Ltd, Singapore (SG)

(72) Inventors: Chi Shang Tai, Singapore (SG); Yayun Ren, Singapore (SG)

(21) Appl. No.: 18/260,124

(22) PCT Filed: Mar. 14, 2022
(86) PCT No.: PCT/SG2022/050128

§ 371 Date: Jun. 30, 2023

(30) Foreign Application Priority Data
Mar. 12, 2021 (US) 63160195

(51) Int. Cl. G16H 30/40; G16H 20/10; G16H 50/20

(52) U.S. Cl. CPC G16H 30/40; G16H 20/10; G16H 50/20

(57) ABSTRACT

A method for assessing nonalcoholic steatohepatitis (NASH) in a liver biopsy sample includes extracting, from the liver biopsy sample, image data indicative of histopathological features. The histopathological features include features selected from the group consisting of fibrosis, inflammation, ballooning and steatosis. The method also includes applying a trained model for assessing a selected histopathological feature to the extracted image data to determine an index associated with said histopathological feature, and determining a degree of NASH based on the determined index. Training the model for assessing the selected histopathological feature includes providing a plurality of training samples and a plurality of validation samples, each sample comprising a graded liver biopsy sample; quantifying parameters of the histopathological features from image data of each of the training samples; selecting a subset of quantified parameters, the subset including one or more parameters of the selected histopathological feature; constructing a model for assessing the selected histopathological feature from the subset of quantified parameters; and validating the constructed model using the validation samples

### CROSS REFERENCE

This application is a national stage entry application under 35 U.S.C. 371 of PCT Patent Application No. PCT/SG2022/050128, filed 14 Mar. 2022, which claims priority to US Provisional Patent Application No. 63/160,195, filed 12 Mar. 2021, the entire contents of each of which are incorporated herein by reference.

### TECHNICAL FIELD

The present disclosure relates broadly, but not exclusively, to methods and systems for assessing nonalcoholic steatohepatitis in a liver biopsy sample.

### BACKGROUND

Nonalcoholic fatty liver disease (NAFLD) covers a pathological spectrum of liver injury characterized by excess fat accumulation within hepatocytes in the absence of harmful alcohol consumption. NAFLD encompasses steatosis (nonalcoholic fatty liver, NAFL), steatohepatitis (nonalcoholic steatohepatitis, NASH), fibrosis and ultimately cirrhosis. Being highly prevalent, it places a substantial burden on global healthcare resources that is predicted to increase further over the next decade. Consequently, there is substantial interest and a need to develop pharmacological therapy.

Although grade (activity) of steatohepatitis varies over time, it is accepted as the underlying driver of fibrogenesis, which in turn determines long-term outcome. Therefore, current regulatory guidance from the US Food and Drug Administration (FDA) and European Medicines Agency (EMA) mandates that drug development should target patients with NASH rather than NAFL, as the latter may be best addressed through lifestyle change. This distinction is key to patient selection for trial enrolment and also serves as one of the surrogate endpoints for drug efficacy assessment. Histological assessment of liver biopsy remains the basis for diagnosing NASH, grading activity and assessing stage of fibrosis. The presence of hepatocellular ballooning is generally considered an essential component in the composite of histological features leading to a diagnosis of NASH as it is thought to represent a form of hepatocyte injury associated with fibrogenesis that is not seen in non-progressive disease.

Two semiquantitative scoring systems have been proposed to aid consistent histopathological interpretation and grading and staging of biopsies: the NASH-Clinical Research Network (CRN) ‘NAFLD Activity Score’ (NAS) and fibrosis stage; and the FLIP/EPOS ‘Steatosis-Activity-Fibrosis’ (SAF) score. Both measure hepatocyte ballooning on a 3-point scale (0-2) but with nuanced differences. It is apparent, however, that the categorical definitions in both semi-quantitative systems may be subject to variation in their interpretation and application. Interobserver variation in pathologists' assessment of grade of activity in general, and ballooning specifically, have been documented.

Computer-assisted tools have been developed to support histopathological assessment, particularly from digitized slide review, with the aim of providing reproducible, objective and standardized evaluation of the significant histopathological features. In one conventional approach, pathologists and artificial intelligence (AI) assess solely fibrosis features in order to diagnose the stage of fibrosis. Similarly, they will focus on steatosis features, ballooning features, inflammation features to diagnose the scores of steatosis, ballooning, and inflammation respectively. However, it has been noted that the convention approach may provide inadequate or inaccurate assessment.

Thus, there is a need to provide methods and devices that can address at least some of the above problems.

### SUMMARY

An aspect of the present disclosure provides a method for assessing nonalcoholic steatohepatitis (NASH) in a liver biopsy sample the method comprising: extracting, from the liver biopsy sample, image data indicative of histopathological features, the histopathological features comprising features selected from the group consisting of fibrosis, inflammation, ballooning and steatosis; applying a trained model for assessing a selected histopathological feature to the extracted image data to determine an index associated with said histopathological feature; and determining a degree of NASH based on the determined index. Training the model for assessing the selected histopathological feature comprises: providing a plurality of training samples and a plurality of validation samples, each sample comprising a graded liver biopsy sample; quantifying parameters of the histopathological features from image data of each of the training samples; selecting a subset of quantified parameters, the subset including one or more parameters of the selected histopathological feature; constructing a model for assessing the selected histopathological feature from the subset of quantified parameters; and validating the constructed model using the validation samples.

There is also disclosed a method for evaluating efficacy of a therapeutic intervention, the method comprising: determining, from a first liver biopsy sample of a subject before the therapeutic intervention, a first degree of NASH using the method as described above; determining, from a second liver biopsy sample of the subject after the therapeutic intervention, a second degree of NASH using the method as described above; and comparing the first degree and second degree to determine efficacy of the therapeutic intervention.

Another aspect of the system for assessing nonalcoholic steatohepatitis (NASH) in a liver biopsy sample, the system comprising a processor; and a computer-readable memory coupled to the processor. The memory has instructions stored thereon that are executable by the processor to: receive image data of the liver biopsy sample indicative of histopathological features, the histopathological features comprising features selected from the group consisting of fibrosis, inflammation, ballooning and steatosis; apply a trained model for assessing a selected histopathological feature to the extracted image data to determine an index associated with said histopathological feature; and determine a degree of NASH based on the determined index. The trained model for assessing the selected histopathological feature comprises: a quantification module for quantifying parameters of the histopathological features from image data of each of a plurality of training samples, each training sample comprising a graded liver biopsy sample; a selection module for selecting a subset of quantified parameters, the subset including one or more parameters of the selected histopathological feature; a construction module for constructing a model for assessing the selected histopathological feature from the subset of quantified parameters; and a validation module for validating the constructed model using a plurality of validation samples, each validation sample comprising a graded liver biopsy sample.

### BRIEF DESCRIPTION OF THE DRAWINGS

Embodiments of the disclosure will be better understood and readily apparent to one of ordinary skill in the art from the following written description, by way of example only, and in conjunction with the drawings, in which:

FIG. 1 is a flow chart illustrating a method for assessing nonalcoholic steatohepatitis (NASH) in a liver biopsy sample according to an example embodiment.

FIG. 2 is a flow chart illustrating a method of forming a trained model for quantitative ballooning assessment according to an example embodiment.

FIG. 3 is a flow chart illustrating a method of forming a trained model for quantitative inflammation assessment according to an example embodiment.

FIG. 4 is a flow chart illustrating a method of forming a trained model for quantitative steatosis assessment according to an example embodiment.

FIG. 5 is a flow chart illustrating a method of forming a trained model for quantitative fibrosis assessment according to an example embodiment.

FIG. 6 shows a schematic diagram of a computer device capable of implementing aspects of the present method and system.

### DETAILED DESCRIPTION

When performing histopathological assessment, it has been noted that fibrosis, inflammation, ballooning and steatosis scores may be inter-connected. For example, for ballooning, identification is related to the fibrosis, steatosis and inflammation next to the ballooning hepatocyte, even though it is not described in the textbook nor was this mentioned in the pathology system like NASH-CRN. The present disclosure provides an approach that utilises all features from ballooning, and (in addition) fibrosis, steatosis, inflammation, to build a model for ballooning assessment (hereinafter also referred to as qBallooning). The performance of qBallooning according to the present disclosure is better than obtained from using only features from ballooning. Similarly, this approach can be applied for quantitative assessment of fibrosis, steatosis and inflammation (hereinafter also referred to as qFibrosis, qSteatosis, and qInflammation respectively), using all the features from Ballooning, Fibrosis, Steatosis, and Inflammation.

Embodiments will be described, by way of example only, with reference to the drawings. Like reference numerals and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “scanning”, “calculating”, “determining”, “applying”, “extracting”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a conventional computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the scope of the disclosure.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM, GPRS, 3G, 4G or 5G mobile telephone systems, as well as other wireless systems such as Bluetooth, ZigBee, Wi-Fi. The computer program when loaded and executed on such a computer effectively results in an apparatus that implements the steps of the preferred method.

The present disclosure may also be implemented as hardware elements. More particularly, in the hardware sense, an element is a functional hardware unit designed for use with other components of elements. For example, an element may be implemented using discrete electronic components, or it can form a portion of an entire electronic circuit such as an Application Specific Integrated Circuit (ASIC) or Field Programmable Gate Array (FPGA). Numerous other possibilities exist. Those skilled in the art will appreciate that the system can also be implemented as a combination of hardware and software elements.

According to various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g. a microprocessor (e.g. a Complex Instruction Set Computer (CISC) processor or a Reduced Instruction Set Computer (RISC) processor). A “circuit”, or “module” may also be a processor executing software, e.g. any kind of computer program, e.g. a computer program using a virtual machine code such as e.g. Java. Any other kind of implementation of the respective functions which may be described in more detail herein may also be understood as a “circuit” in accordance with an alternative embodiment.

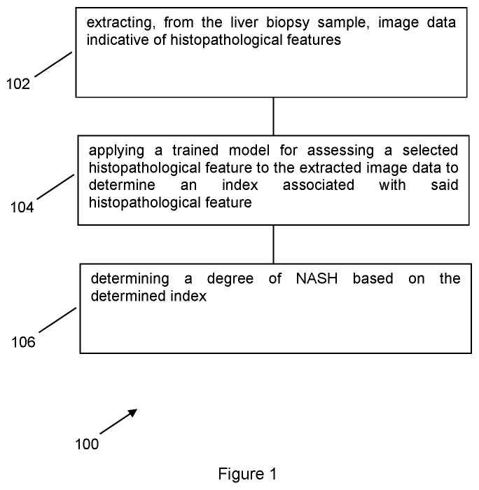

FIG. 1 is a flow chart 100 illustrating a method for assessing nonalcoholic steatohepatitis (NASH) in a liver biopsy sample according to an example embodiment. At step 102, image data indicative of histopathological features is extracted from the liver biopsy sample. The histopathological features include features selected from the group consisting of fibrosis, inflammation, ballooning and steatosis. At step 104, a trained model for assessing a selected histopathological feature is applied to the extracted image data to determine an index associated with said histopathological feature. For example, a qBallooning model may be used to determine a ballooning index. At step 106, a degree of NASH is determined based on the determined index.

The model for the selected histopathological feature may be trained by providing a plurality of training samples and a plurality of validation samples, each sample comprising a graded liver biopsy sample; quantifying parameters of the histopathological features from image data of each of the training samples; selecting a subset of quantified parameters, the subset including one or more parameters of the selected histopathological feature; constructing a model for the selected histopathological feature from the subset of quantified parameters; and validating the constructed model using the validation samples.

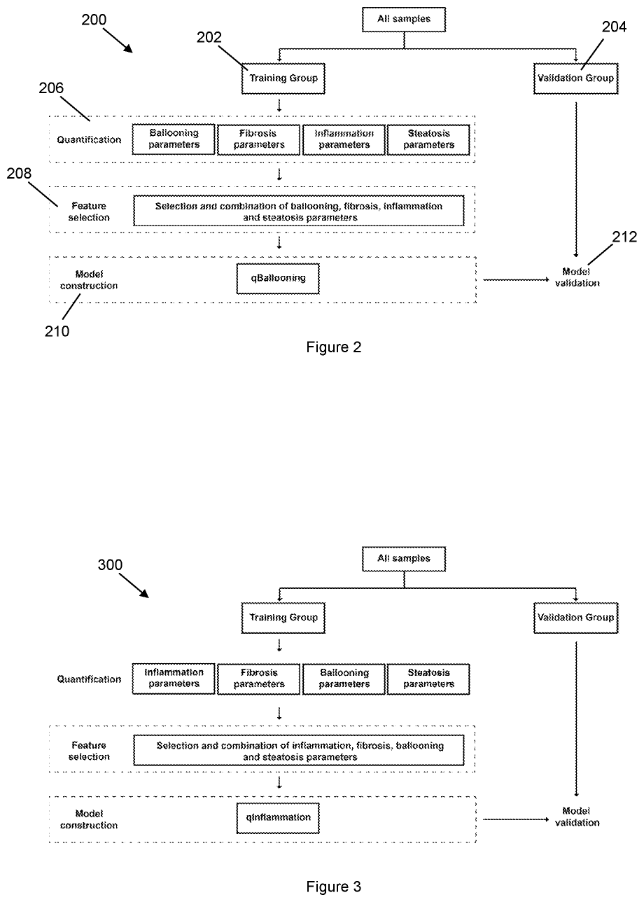

FIG. 2 is a flow chart 200 illustrating a method of forming a trained model for quantitative ballooning assessment (qBallooning) according to an example embodiment.

During sample preparation stage, liver histology from all liver biopsy samples is assessed by expert pathologists and the scores are determined according to the NASH CRN system (steatosis scores 0-3, ballooning scores 0-2, and lobular inflammation scores 0-3). Liver fibrosis is staged as 0-4 using the NASH CRN scoring system, with 4 indicating cirrhosis. In other words, each liver biopsy sample is graded according to established scoring systems.

All samples are then divided into two groups randomly: a training group 202 and a validation group 204. The qBallooning model is built based on the samples in the training group 202 and validated on the samples in the validation group 204. For example, a total of 300 samples are used in one implementation, and the samples are divided into the training group 202 and validation group 204 according to stratified randomization. For the samples of each fibrosis stage, two thirds are randomly selected to be assigned to the training group and the remaining third to the validation group. Thus, there are 200 samples in the training group 202 and 100 samples in the validation group 204.

Next, images are acquired on unstained sections of the above NASH samples, using a Genesis system (Histoindex Pte. Ltd, Singapore), in which second harmonic generation (SHG) microscopy is used to visualize collagen, and the other cell structures are visualized using two-photon excited fluorescence (TPEF) microscopy. From the SHG/TPEF images, parameters are quantified at step 206. For example, n1 ballooning parameters, n2 fibrosis parameters, n3 inflammation parameters and n4 steatosis parameters are quantified using the image analysis algorithm for each sample. Total N (n1+n2+n3+n4) parameters are quantified.

Parameter selection is performed at step 208 to reduce the dimensionality of data by selecting only a subset of quantified parameters. In one embodiment, sequential feature selection is used to identify features which are meaningful or significant. In the procedure of sequential feature selection, a linear regression model is used whereby the criterion is a residual sum of squares and the search algorithm is a sequential forward selection. With the feature selection method, N1 parameters will be selected from the N parameters to evaluate the ballooning grades read by pathologists. The N1 parameters may include one or more kinds of parameters, such as all N1 parameters are ballooning parameters, or N1 parameters include ballooning and fibrosis parameters. It will be appreciated that other combinations or permutations are possible, but there should be one or more ballooning parameters when building a qBallooning model.

Based on the selected N1 parameters, the qBallooning model can be built at step 210, for example, by using the linear regression method. Each sample from the training group 202 has a corresponding qBallooning continuous value based on the constructed qBallooning model. In other word, a set of continuous values may be generated from the training group 202. Further, the cut-off values for determining each corresponding NASH grade are determined by Youden's index.

At step 212, the constructed qBallooning model is validated on the samples in validation group 204. Based on the N1 parameters and the constructed qBallooning model, a qBallooning continuous value can be calculated for each validation sample. The performance of qBallooning model can be evaluated by the correlation of the qBallooning continuous value (i.e. effectively the grade assessed by the model) and the ballooning grade assessed by a pathologist.

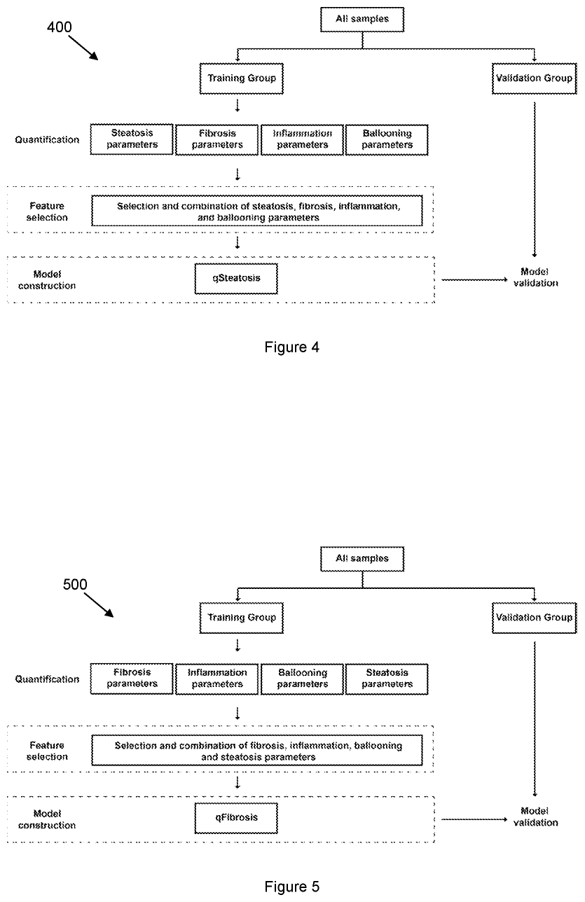

Models for quantitatively assessing other histopathological features can be similarly built. FIG. 3 is a flow chart 300 illustrating a method of forming a trained model for quantitative inflammation assessment (qInflammation) according to an example embodiment. FIG. 4 is a flow chart 400 illustrating a method of forming a trained model for quantitative steatosis assessment (qSteatosis) according to an example embodiment. FIG. 5 is a flow chart 500 illustrating a method of forming a trained model for quantitative fibrosis assessment (qFibrosis) according to an example embodiment. Similar to the qBallooning model, all ballooning, inflammation, steatosis and fibrosis parameters are initially quantified. To build the qInflammation model, the selected subset of quantified parameters includes one or more inflammation parameters. To build the qSteatosis model, the selected subset of quantified parameters includes one or more steatosis parameters. To build the qFibrosis model, the selected subset of quantified parameters includes one or more fibrosis parameters. For brevity, these drawings are not described again here.

### Example

Liver biopsy samples were taken from two randomized controlled trials. The ‘development’ cohort comprised ten trial-entry screening biopsies selected to encompass a spectrum of NAFLD grade/stage from non-NASH NAFL (i.e. no ballooning, B0) to NASH with marked ballooning (B2) and moderate fibrosis (F2-3). Twenty-two cases with paired biopsies were selected as an independent ‘test’ cohort for the qBallooning algorithm. Digitised images of the haematoxylin and eosin (H&E) stained liver tissue sections were acquired using the Aperio Digital Pathology Imaging Systems (Leica Biosystems).

All imaging of unstained sections was conducted by trained technicians on identical equipment (Genesis™ system HistoIndex Pte. Ltd., Singapore) according to a standardized operating procedure.

Annotated ballooned cells on the 10 pre-selected digital H&E slides made by the pathologists were recorded and used to generate the “ground truth” of training sets on the corresponding SHG/TPEF images for the artificial intelligence algorithm. Suitable candidates of ballooned hepatocytes on the TPEF channel were identified using traditional image analysis methods, including image segmentation, morphological processing, and watershed algorithm as previously described.

A total of 45 ballooning parameters were established and quantified, including the number of ballooned hepatocytes, the area of ballooned hepatocytes and the area of “collagen area” around the ballooned hepatocytes. Subsequently, paired digitized liver biopsy slides (n=44) from the development set were used to establish a qBallooning index, which can indicate the degree of ballooning. Images were processed and analysed using MATLAB 8.3 (The MathWork, USA).

From an overall data set of 45 features, the qBallooning index was established based on 7 parameters, including 6 ballooned cell parameters: total perimeter of ballooned hepatocytes per unit tissue area, variance in distance between ballooned hepatocytes and the nearest ballooned hepatocytes, average distance between ballooned hepatocytes and the nearest ballooned hepatocytes, average number of ballooned hepatocytes within 100 μm of a ballooned hepatocyte, variance in number of ballooned hepatocytes within 100 μm of a ballooned hepatocyte; and 1 collagen parameter: total collagen area around ballooned hepatocytes per unit tissue area.

When the qBallooning model was trained using the full atlas of 1,188 cells identified as ballooned by at least one pathologist, 346 cells were flagged by the algorithm of which 198 cells (57%) had also been identified by the pathologists. Performance of the qBallooning algorithm may be further tuned according to the number of pathologists providing concordance that were used to train it.

To establish proof-of-principle whether qBallooning was sensitive to change in the context of NASH clinical trials, selected samples formed an independent test cohort. Samples were chosen from patients that, irrespective of treatment arm, at the end of the study were reported by the trial pathologist to have either at least 1-point NASH-CRN ballooning score reduction (‘improvers’), or no ballooning score reduction (‘non-improvers’).

Amongst ‘improvers’ that were judged to show a reduction in ballooned hepatocytes by the trial pathologist, relative to the baseline biopsy qBallooning detected a median (lower quartile, upper quartile) 79%(−89%, −19%) reduction in number of ballooned hepatocytes. In contrast, a mean 77%(−46%, 143%) increase in ballooned hepatocytes was detected in ‘non-improvers’ at the end of the study (p=0.038).

Depending on the clinical context the algorithm may be calibrated differently for diagnosis or for the detection of clinically relevant temporal changes, for instance, in therapeutic trials. The method as disclosed demonstrates it has the capacity to detect change in ballooning deemed relevant to identify drug-induced histological changes. For example, the method can be applied to a biopsy sample from a subject before a therapeutic intervention and to another sample from the same subject after the therapeutic intervention. The results can be compared to determine the efficacy of the therapeutic intervention. In other words, the present disclosure provides an application of artificial intelligence that offers a potential assistive technology that may complement human pathology where there is a need for reproducible cut-points that determine go/no-go decisions in drug development.

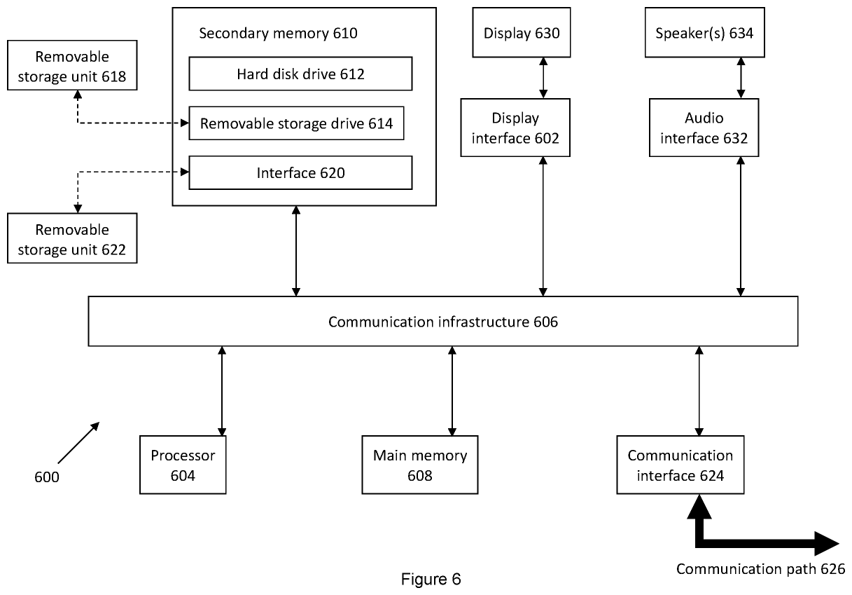

FIG. 6 depicts an exemplary computing device 600, hereinafter interchangeably referred to as a computer system 600, where one or more such computing devices 600 may be used for at least some steps of the present method. For example, a system that performs the present method of assessing NASH in a liver biopsy sample may include the computing device 600 connected to an image data acquisition device, such as a second harmonic generation (SHG) microscope and/or a two photon excitation fluorescence (TPEF) microscope (not shown). Alternatively, the system may include a stand-alone computing device 600 and receive image data that has been acquired separately. The following description of the computing device 600 is provided by way of example only and is not intended to be limiting.

As shown in FIG. 6, the example computing device 600 includes a processor 604 for executing software routines. Although a single processor is shown for the sake of clarity, the computing device 600 may also include a multi-processor system. The processor 604 is connected to a communication infrastructure 606 for communication with other components of the computing device 600. The communication infrastructure 606 may include, for example, a communications bus, cross-bar, or network.

The computing device 600 further includes a main memory 608, such as a random access memory (RAM), and a secondary memory 610. The secondary memory 610 may include, for example, a hard disk drive 612 and/or a removable storage drive 614, which may include a floppy disk drive, a magnetic tape drive, an optical disk drive, or the like. The removable storage drive 614 reads from and/or writes to a removable storage unit 618 in a well-known manner. The removable storage unit 618 may include a floppy disk, magnetic tape, optical disk, or the like, which is read by and written to by removable storage drive 614. As will be appreciated by persons skilled in the relevant art(s), the removable storage unit 618 includes a computer readable storage medium having stored therein computer executable program code instructions and/or data.

In an alternative implementation, the secondary memory 610 may additionally or alternatively include other similar means for allowing computer programs or other instructions to be loaded into the computing device 600. Such means can include, for example, a removable storage unit 622 and an interface 620. Examples of a removable storage unit 622 and interface 620 include a program cartridge and cartridge interface (such as that found in video game console devices), a removable memory chip (such as an EPROM or PROM) and associated socket, and other removable storage units 622 and interfaces 620 which allow software and data to be transferred from the removable storage unit 622 to the computer system 600.

The computing device 600 also includes at least one communication interface 624. The communication interface 624 allows software and data to be transferred between computing device 600 and external devices via a communication path 626. In various embodiments of the disclosure, the communication interface 624 permits data to be transferred between the computing device 600 and a data communication network, such as a public data or private data communication network. The communication interface 624 may be used to exchange data between different computing devices 600 which such computing devices 600 form part an interconnected computer network. Examples of a communication interface 624 can include a modem, a network interface (such as an Ethernet card), a communication port, an antenna with associated circuitry and the like. The communication interface 624 may be wired or may be wireless. Software and data transferred via the communication interface 624 are in the form of signals which can be electronic, electromagnetic, optical or other signals capable of being received by communication interface 624. These signals are provided to the communication interface via the communication path 626.

As shown in FIG. 6, the computing device 600 further includes a display interface 602 which performs operations for rendering images to an associated display 630 and an audio interface 632 for performing operations for playing audio content via associated speaker(s) 634.

As used herein, the term “computer program product” may refer, in part, to removable storage unit 618, removable storage unit 622, a hard disk installed in hard disk drive 612, or a carrier wave carrying software over communication path 626 (wireless link or cable) to communication interface 624. Computer readable storage media refers to any non-transitory tangible storage medium that provides recorded instructions and/or data to the computing device 600 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computing device 600. Examples of transitory or non-tangible computer readable transmission media that may also participate in the provision of software, application programs, instructions and/or data to the computing device 600 include radio or infra-red transmission channels as well as a network connection to another computer or networked device, and the Internet or Intranets including e-mail transmissions and information recorded on Websites and the like.

The computer programs (also called computer program code) are stored in main memory 608 and/or secondary memory 610. Computer programs can also be received via the communication interface 624. Such computer programs, when executed, enable the computing device 600 to perform one or more features of embodiments discussed herein. In various embodiments, the computer programs, when executed, enable the processor 604 to perform features of the above-described embodiments. Accordingly, such computer programs represent controllers of the computer system 600.

Software may be stored in a computer program product and loaded into the computing device 600 using the removable storage drive 614, the hard disk drive 612, or the interface 620. Alternatively, the computer program product may be downloaded to the computer system 600 over the communications path 626. The software, when executed by the processor 604, causes the computing device 600 to perform functions of embodiments described herein.

It is to be understood that the embodiment of FIG. 6 is presented merely by way of example. Therefore, in some embodiments one or more features of the computing device 600 may be omitted. Also, in some embodiments, one or more features of the computing device 600 may be combined together. Additionally, in some embodiments, one or more features of the computing device 600 may be split into one or more component parts.

It will be appreciated that the elements illustrated in FIG. 6 function to provide means for performing the various functions and operations of the servers as described in the example embodiments.

In an implementation, a server may be generally described as a physical device comprising at least one processor and at least one memory including computer program code. The at least one memory and the computer program code are configured to, with the at least one processor, cause the physical device to perform the requisite operations.

It will be appreciated by a person skilled in the art that numerous variations and/or modifications may be made to the present disclosure as shown in the specific embodiments without departing from the scope of the disclosure as broadly described. For example, the number and composition of the quantified parameters may be varied. The sensitivity of the model may be adjusted depending on the concordance of pathologists who grade the samples used to train and validate the model. The present embodiments are, therefore, to be considered in all respects to be illustrative and not restrictive.

1. A method for assessing nonalcoholic steatohepatitis (NASH) in a liver biopsy sample, the method comprising:
extracting, from the liver biopsy sample, image data indicative of histopathological features, the histopathological features comprising features selected from the group consisting of fibrosis, inflammation, ballooning and steatosis;
applying a trained model for assessing a selected histopathological feature to the extracted image data to determine an index associated with said histopathological feature; and
determining a degree of NASH based on the determined index,
wherein training the model for assessing the selected histopathological feature comprises:
providing a plurality of training samples and a plurality of validation samples, each sample comprising a graded liver biopsy sample;
quantifying parameters of the histopathological features from image data of each of the training samples;
selecting a subset of quantified parameters, the subset including one or more parameters of the selected histopathological feature;
constructing a model for assessing the selected histopathological feature from the subset of quantified parameters; and
validating the constructed model using the validation samples.

2. The method as claimed in claim 1, wherein the image data of each of the training samples is obtained by second harmonic generation (SHG) microscopy and/or two photon excitation fluorescence (TPEF) microscopy.

3. The method as claimed in claim 1, wherein selecting the subset of quantified parameters comprises a sequential feature selection.

4. The method as claimed in claim 3, wherein the sequential feature selection uses a linear regression model, wherein a criterion of the linear regression model comprises a residual sum of squares and a search algorithm of the linear regression model comprises a sequential forward selection.

5. The method as claimed in claim 1, wherein constructing the model for assessing the selected histopathological feature from the subset of quantified parameters comprises a linear regression procedure.

6. The method as claimed in claim 5, wherein the plurality of training samples have a corresponding plurality of continuous values based on the model for assessing the selected histopathological feature, and wherein the method further comprises determining cut-off values of each NASH grade by Youden's index.

7. The method as claimed in claim 6, wherein validating the model using the validation samples comprises:
for each validation sample, determining a quantitative value using the constructed model for assessing the selected histopathological feature, thereby determining the corresponding NASH grade; and
correlating the determined NASH grade with a grade provided by a pathologist.

8. A method for evaluating efficacy of a therapeutic intervention, the method comprising:
determining, from a first liver biopsy sample of a subject before the therapeutic intervention, a first degree of NASH using the method as claimed in any one of the preceding claims;
determining, from a second liver biopsy sample of the subject after the therapeutic intervention, a second degree of NASH using the method as claimed in any one of the preceding claims; and
comparing the first degree and second degree to determine efficacy of the therapeutic intervention.

9. A system for assessing nonalcoholic steatohepatitis (NASH) in a liver biopsy sample, the system comprising:
a processor; and
a computer-readable memory coupled to the processor and having instructions stored thereon that are executable by the processor to:
receive image data of the liver biopsy sample indicative of histopathological features, the histopathological features comprising features selected from the group consisting of fibrosis, inflammation, ballooning and steatosis;
apply a trained model for assessing a selected histopathological feature to the extracted image data to determine an index associated with said histopathological feature; and
determine a degree of NASH based on the determined index,
wherein the trained model for assessing the selected histopathological feature comprises:
a quantification module for quantifying parameters of the histopathological features from image data of each of a plurality of training samples, each training sample comprising a graded liver biopsy sample;
a selection module for selecting a subset of quantified parameters, the subset including one or more parameters of the selected histopathological feature;
a construction module for constructing a model for assessing the selected histopathological feature from the subset of quantified parameters; and
a validation module for validating the constructed model using a plurality of validation samples, each validation sample comprising a graded liver biopsy sample.

10. The system as claimed in claim 9, wherein the image data of each of the training samples comprises data from a second harmonic generation (SHG) microscope and/or a two-photon excitation fluorescence (TPEF) microscope.

11. The system as claimed in claim 9, wherein the selection module is configured to select the subset of quantified parameters by a sequential feature selection.

12. The system as claimed in claim 11, wherein the sequential feature selection uses a linear regression model, wherein a criterion of the linear regression model comprises a residual sum of squares and a search algorithm of the linear regression model comprises a sequential forward selection.

13. The system as claimed in claim 9, wherein the construction module is configured to construct the model for assessing the selected histopathological feature from the subset of quantified parameters by a linear regression procedure.

14. The system as claimed in claim 13, wherein the plurality of training samples have a corresponding plurality of continuous values based on the model for assessing the selected histopathological feature, and wherein cut-off values of each NASH grade are determined by Youden's index.

15. The system as claimed in claim 14, wherein the validation module is configured to:
for each validation sample, determine a quantitative value using the constructed model for assessing the selected histopathological feature, thereby determining the corresponding NASH grade; and
correlate the determined NASH grade with a grade provided by a pathologist.
